(19) United States
(12) Patent Application Publication
(10) Pub. No.: US 2022/0362176 A1
(43) Pub. Date: Nov. 17, 2022

(54) CATIONIC ANTIMICROBIAL OLIGO-GUANIDINIUM DENDRIMERS AND COMPOSITIONS

(71) Applicant: Hyun Ho Chung, Fullerton, CA (US)

(72) Inventor: Hyun Ho Chung, Fullerton, CA (US)

(21) Appl. No.: 17/744,470

(22) Filed: May 13, 2022

Related U.S. Application Data
(60) Provisional application No. 63/188,621, filed on May 14, 2021.

(51) Int. Cl. A61K 31/155; C07C 277/08; A61P 31/04; A01N 47/44; A01P 1/00; A61K 47/55

(52) U.S. Cl. CPC A61K 31/155; C07C 277/08; A61P 31/04; A01N 47/44; A01P 1/00; A61K 47/552

(57) ABSTRACT

A new synthesized non-peptide cationic oligo-guanidinium dendrimers containing a number of positive charges, 6, 9, 12, and 18 for use as antimicrobial agents and a method for synthesizing non-peptide cationic oligo-guanidinium dendrimers G12 and G18. These designed and synthesized dendrimers exhibit antimicrobial activities as well as biofilm disrupting activity against various pathogens such as Gram-negative bacteria, Gram-positive bacteria, and fungi. The application of these dendrimers are very broad, which includes application to pharmaceutical compounds that are covalently or non-covalently complexed. These combinations are important in helping the body reach the proper immune balance required for maximized function and/or optimal health.

### CROSS-REFERENCE TO RELATED APPLICATIONS

The present application claims the priority of U.S. Provisional Patent Application Ser. No. 63/188,621 May 14, 2021, which application is incorporated in its entirety herein by reference.

### BACKGROUND OF THE INVENTION

The invention relates to compositions of cationic antimicrobial oligo-guanidinium dendrimers, to methods for producing and using the compositions, and to articles comprising the compositions. The antimicrobial oligo-guanidinium dendrimers of the present invention are useful in pharmaceutical, healthcare, medical devices, industrial, food, agricultural, and personal care applications.

Over many decades, antibiotics have proved essential for the therapy of different pathogen infections. However, the increase in microbial resistance to antibiotics due to their excess of use during recent years means that new therapeutic strategies are necessary.

In this context, the World Health Organization (WHO) issued a public health warning in 2014, which stated that this is not a problem that humanity will face in the future but is something that is happening today in the treatment of certain infections resistant to conventional antibiotics. Thereby, the increasing incidence of microbial infections resistant to antibiotics is one of the greatest challenges that modern medicine faces today.

In this regard, a great number of cationic peptides that are the first natural barrier against external pathogens have been discovered and characterized. These cationic peptides can be found in mammals, insects, plants, and the skins of some amphibians.

The two main characteristics of these cationic peptides are that they have positive charges, ranging from 2 to 9, and that they fold in an amphipathic helical conformation, with two well-defined hydrophilic and hydrophobic faces.

Although the alpha conformation of the peptides is the most common, it has been reported that cationic peptides with beta-sheet conformation can also show antibacterial activity.

The main limitation for developing these cationic peptides as a therapeutic agent is the lack of detailed knowledge about their action mechanism at the molecular level.

In line with the mechanism of action of these antimicrobial peptides, recent studies show that the action mechanism is based on the destabilization of the cell membrane of Gram-negative and Gram-positive bacteria and fungus. Thus, these peptides exert their lytic activity against pathogens by electrostatic interactions with the negative charge of the microbial cell surfaces and subsequent pathogen membrane disruption. Hence, the positive charge of the cationic peptide seems to be a crucial aspect that needs to be considered in the discrimination process between the pathogen and host cells.

In this context, the positive charge of these peptides, together with the amphipathic nature of their alpha helical conformation, seems to be key aspects for their antimicrobial activity. In addition, the number of positive charges of these peptides is a critical parameter in their antimicrobial activity. Generally, an increase in the positive charge enhances the antimicrobial activity of these cationic peptides, any further enhanced antimicrobial activity above 9 positive charges is negligible.

Therefore, the development of novel therapeutic agents that could overcome the resistance to standard antibiotics seems to be crucial for continuing the fight against diseases. Among novel treatments, small cationic peptides have shown a great potential as a new generation of antibiotics. Increased knowledge of the nature and action mechanism of natural cationic peptides has enabled new synthetic peptides to be produced and tested in clinical trials. These synthetic peptides have shown activity when they were used in animals as models of infections by a variety of pathogens.

However, despite the great expectations in this field, clinical trials for these synthetic peptides have been limited, and none has been approved for use in humans to date, due to their toxicity, poor bio-availability, stability and antigenicity.

### BRIEF SUMMARY OF THE INVENTION

To overcome all the problems associated with the use of these cationic peptides as new antibiotics, new non-peptide cationic oligo-guanidinium dendrimers containing a number of positive charges, 6, 9, 12, and 18 have been synthesized as candidates for use as antimicrobial agents. Recently, these oligo-guanidinium dendrimers have been shown to have a strong antibacterial effect against a panel of pathogenic bacteria including Methicillin-Resistant Staphylococcus Aureus (MRSA).

The present invention describes the design and synthesis of novel nonpeptidic oligo-guanidinium dendrimer compounds, compositions and their use as antimicrobial agents. Previous invention (WO 2004/009665 A3) described dendritic oligoguanidine compounds synthesis and their use as molecular translocators for drug delivery. These dendrimers exhibit antimicrobial activities, as well as biofilm, disrupting activity against various pathogens such as Gram-negative bacteria, Gram-positive bacteria, and fungi and relates to antimicrobial cationic oligo-guanidinium dendrimers containing a number of positive charges, 6, 9, 12 and 18.

The invention also relates to antimicrobial cationic oligo-guanidinium dendrimers as described above, in which pharmaceutical compounds are covalently or non-covalently complexed. These combinations are important in helping the body reach the proper immune balance required for maximized function and/or optimal health.

The invention also relates to the method for the synthesis of G12 nonpeptidic oligo-guanidinium dendrimer compound.

The invention also relates to the method for the synthesis of G18 nonpeptidic oligo-guanidinium dendrimer compound.

### BRIEF DESCRIPTION OF THE SEVERAL VIEWS OF THE DRAWING

FIG. 1 displays G6, which comprises of 6 oligo-guanidinium head groups according to the present invention.

FIG. 2 displays G9, which comprises of 9 oligo-guanidinium head groups.

FIG. 3 displays Synthesis of Pyridyl disulfide derivatives of G6 for the preparation of G12, which comprises of 12 oligo-guanidinium head groups.

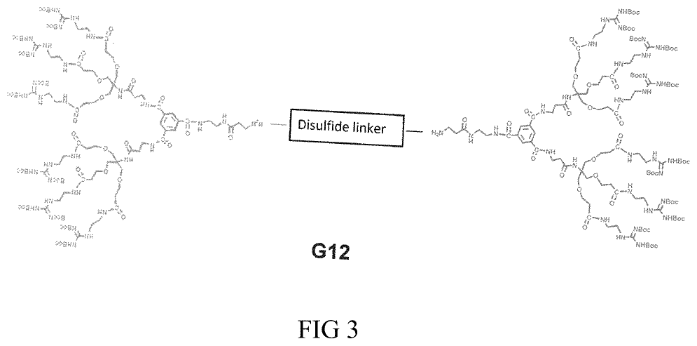

FIG. 4 displays Synthesis of Pyridyl disulfide derivatives of G9 for the preparation of G18, which comprises of 18 oligo-guanidinium head groups.

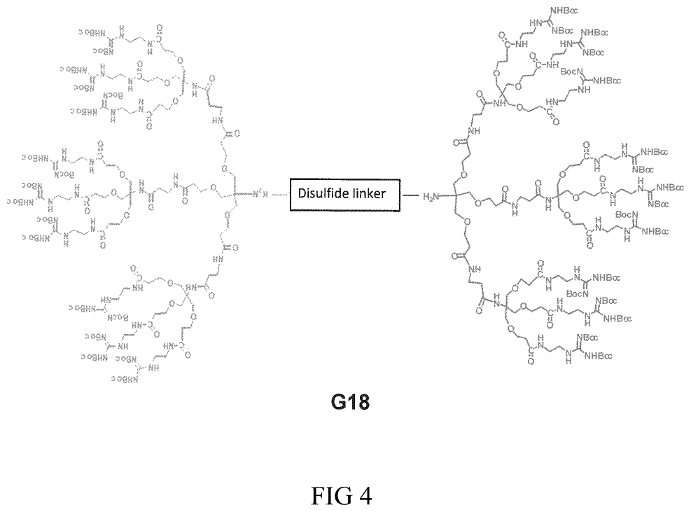

FIG. 5 displays an example of the antibacterial effect of the invention.

FIG. 6 displays the method for the production of G12.

FIG. 7 displays the method for the production of G18.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

Effectiveness as an Antibacterial Agent

The novel nonpeptidic cationic oligo-guanidinium dendrimers according to the invention has been designed and synthesized such that its antibacterial properties make it useful as antibacterial agent for the prevention and therapeutical treatment of infections due to microbial pathogens in human, animal and also vegetables which constitutes an additional object of the invention.

The novel non-peptidic cationic oligo-guanidinium dendrimers according to the invention is especially useful against Gram-positive bacteria growing under aerobic or anaerobic conditions. Such drugs are useful against bacteria of the genus Staphylococcus, more specifically S. aureus and coagulase-negative staphylococci like S. epidermidis and S. saprophyticus (including multidrug-resistant strains such as methicillin-resistant staphylococci, vancomycin intermediate and vancomycin resistant Staphylococcus aureus), Enterococcus (including E. faecalis and E. faecium including vancomycin resistant isolates), Streptococcus (including S. pneumonia, S. agalactiae, S. pyogenes, and streptococci of the viridans group).

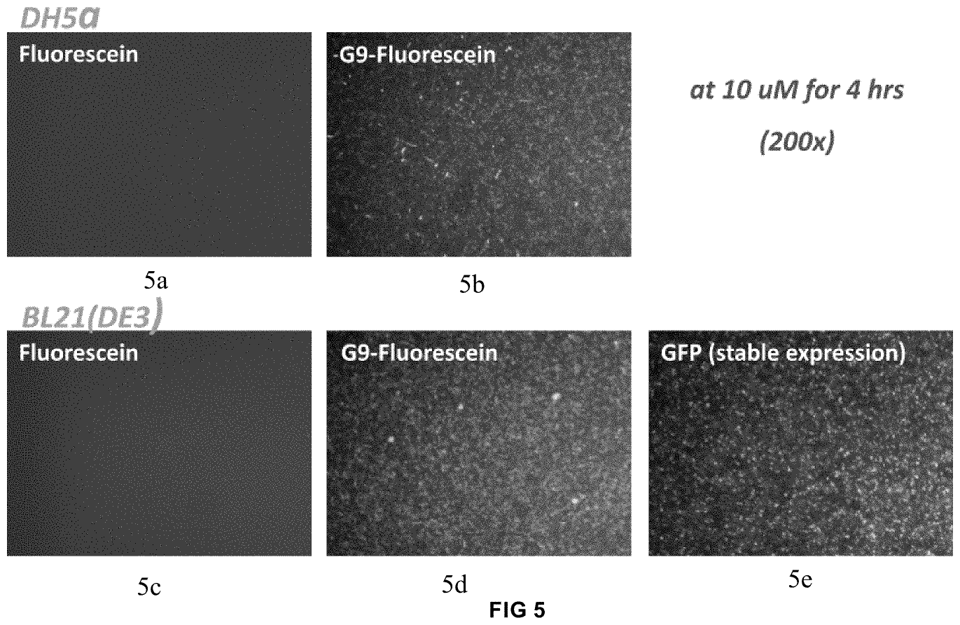

An example of the antibacterial effect as described above is shown in FIG. 5. Transducing activities of dendrimers onto bacterial cells were analyzed using DH5α (See 5a in FIG. 5) and BL21(DE3) (See 5c in FIG. 5). Bacterial cells were prepared in 3 mL of Luria Broth (LB) media and harvested at the log phase of cell growth. Harvested cells were then re-suspended in 200 μl of fresh LB media. To test the transducing activity, the cells were mixed with equal volume of 20 μM of Fluorescein-labeled dendrimers (ex, G9-FL, ref. listed) (See 5b and 5d in FIG. 5), and 20 μM Of Fluorescein compound (as the control) in PBS buffer. The mixtures were incubated for 4 hours at room temperature, and then washed twice with 500 μl of PBS. The samples were re-suspended in 40 μl of PBS and transfer onto 96-well plate. The fluorescence images were taken under the fluorescence microscope. BL21(DE3), stably expressing green fluorescence protein was also prepared as an extra control. (ref. listed) (See 5e in FIG. 5)

Use as Crop Protection

In addition to the above described uses, the cationic oligo-guanidinium dendrimers according to the invention can also be used in the crop protection against plants pathogens. One can mention for example control of Phytophthora blight infection caused by Phytophtora in red pepper.

Use as Pharmaceutical Compositions

The invention also relates to pharmaceutical compositions comprising, as active principle, of a therapeutically effective amount of at least one cationic oligo-guanidinium dendrimers according to the invention. In the compositions of the invention, the active principle can be in association with a pharmaceutically acceptable carrier or excipient.

The pharmaceutical compositions according to the invention are advantageously formulated to be administered under oral, topical, transdermal, sublingual, rectal, parenteral including intravenous, intramuscular, intraperitoneal and subcutaneous routes, with individual doses appropriate for the patient to be treated. The preferred routes are transdermal routes.

The compositions according to the invention can be solid, liquid including solutions, emulsions or suspensions, or in the form of a gel/cream and be presented in the pharmaceutical forms commonly used in human medicine, such as plain or sugar-coated tablets, gelatin capsules, granules, suppositories, injectable preparations, ointments, creams, and gels; all of which are prepared according to the customary methods. The active ingredient/s can be incorporated using excipients which are customarily used in these pharmaceutical compositions, such as talc, gum arabic, lactose, starch, magnesium stearate, aqueous or non-aqueous vehicles, fatty substances of animal or vegetable origin, paraffin derivatives, glycols, various wetting agents, dispersants or emulsifiers, and preservatives. These compositions can in particular be presented in the form of a powder intended to be dissolved or suspended extemporaneously in an appropriate vehicle, for example, non-pyrogenic sterile water.

The dose of the cationic oligo-guanidinium dendrimers according to the invention administered varies according to the condition to be treated, the patient in question and the administration route. It can, for example, be comprised between 10 mg and 10 g per day for an adult.

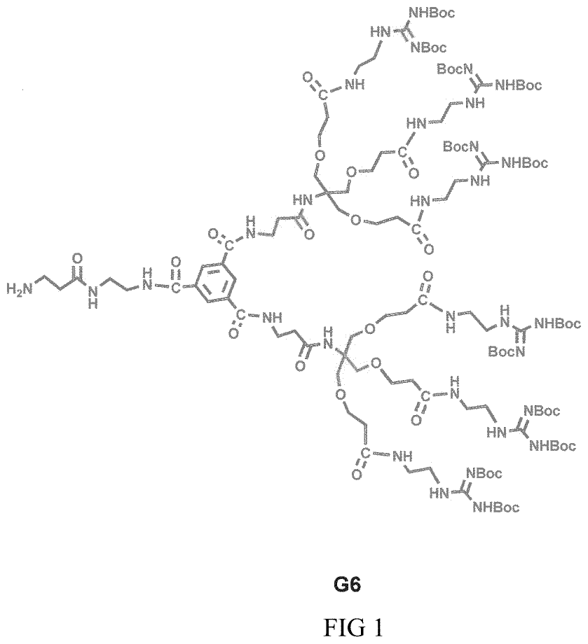

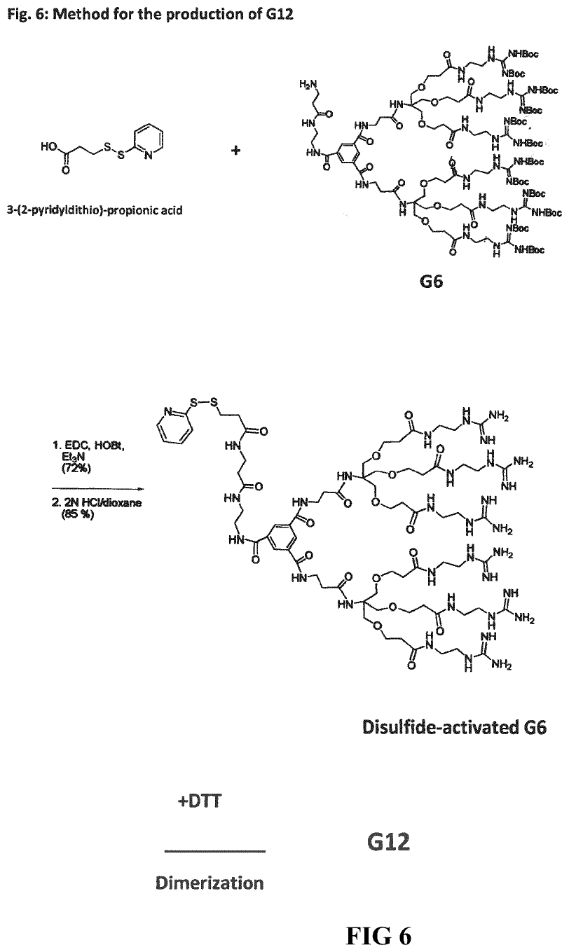

The invention also includes method for the synthesis of G12, as shown on FIG. 6, nonpeptidic oligo-guanidinium dendrimer compound. In the synthesis of G12 the 3-(2-pyridyldithio)-propionic acid was attached to the free amine of G6, as shown on FIG. 1. Deprotection by cleaving the Boc groups from the guanidines afforded the disulfide-activated G6 in good overall yield. G12 was then synthesized by dimerization of disulfide-activated G6 in aqueous solution with Dithiothreitol (DTT).

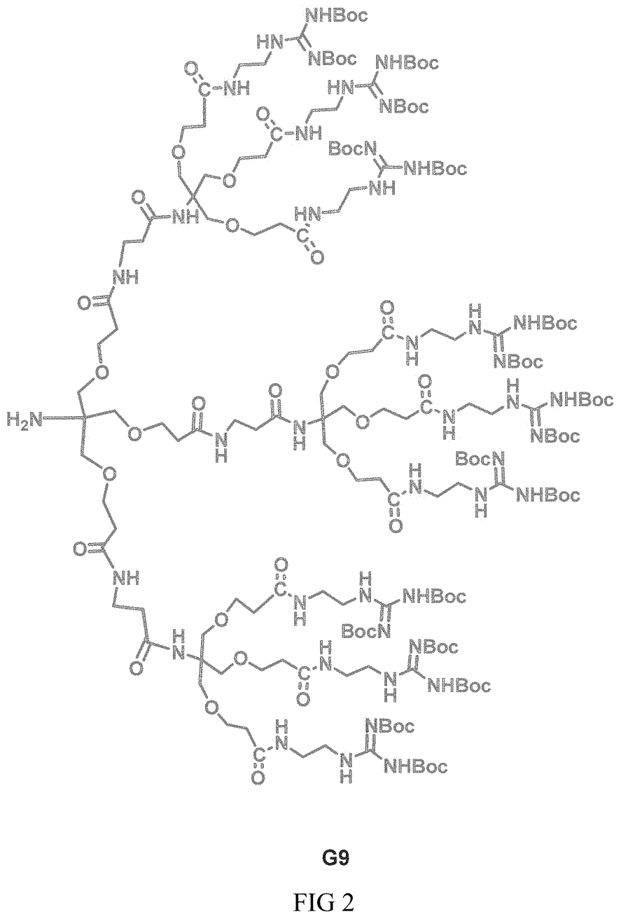

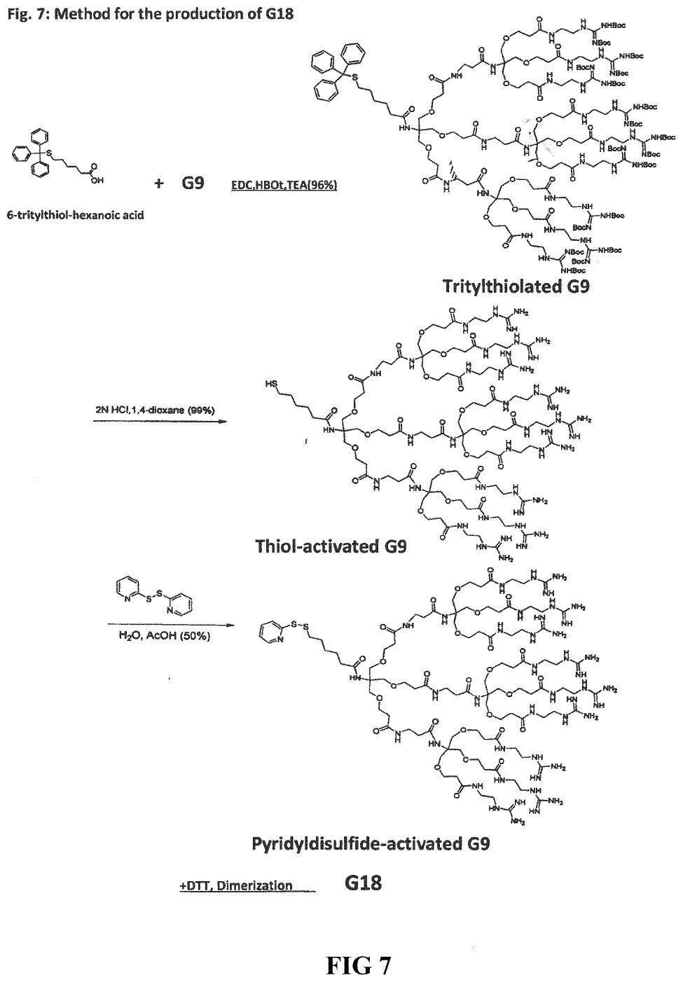

The invention also includes the method for the synthesis of G18, as shown on FIG. 7, nonpeptidic oligo-guanidinium dendrimer compound. In the synthesis for G18 tritylthiolated G9, as shown on FIG. 2, was prepared by adding 6-tritylthiol-hexanoic acid to the free amine of G9. The Boc groups and the trityl groups were then removed in one step by addition of 2 N HCl. Pyridyldisulfide-activated G9 was then prepared by using dipyridyldisulfide in mild acidic aqueous solution. Final product G18 was then prepared by dimerization of the pyridyldisulfide-activated G9 and subsequent purification.

I claim:

1. An antibiotic molecule, comprising:
dendrimers comprising non-peptide cationic oligo-guanidinium containing a number of positive charges, 6, 9, 12, and 18 and headgroups.

2. The antibiotic molecule in claim 1, wherein the dendrimers are covalently or non-covalently complexed with pharmaceutical compounds.

3. The antibiotic molecule in claim 1, further comprising a Disulfide linker.

4. A method for the production of G12, comprising:
a. attaching a free amine of G6 to a 3-(2-pyridyldithio)-propionic acid;
b. deprotection by cleaving the Boc groups from the guanidines; and
c. synthesizing G12 by dimerization of disulfide-activated G6 in aqueous solution with Dithiothreitol (DTT).

5. A method for the production of G18, comprising:
a. add 6-tritylthiol-hexanoic acid to a free amine of G9;
b. Boc groups and trityl groups are removed in one step by adding of 2 N HCL;
c. pyridyldisulfide-activated G9 is then prepared by using dipyridyldisulfide in mild acid aqueous solution; then
d. dimerization of the pyridyldisulfide-activated G9 and subsequent purification.
